(19) United States
(12) Patent Application Publication
(10) Pub. No.: US 2011/0184302 A1
(43) Pub. Date: Jul. 28, 2011

(54) MEDICAL DETECTION DEVICE FOR THE DETECTION OF SLEEP APNEA AND/OR SLEEP HYPOPNEA

(76) Inventors: Johannes Eschler, Ditzingen (DE); Thomas Baehren, Remseck A.N. (DE); Ulrich Ladstaetter, Weinstadt (DE); Angelika Pauls-Von Heyden, Stuttgart (DE)

(21) Appl. No.: 12/736,186

(22) PCT Filed: Jan. 30, 2009
(86) PCT No.: PCT/EP2009/051088

§ 371 Date: Mar. 24, 2011

(30) Foreign Application Priority Data
Mar. 17, 2008 (DE) 10 2008 014 652.8

(51) Int. Cl. A61B 5/08

(52) U.S. Cl. 600/529

(57) ABSTRACT

A medical detection device for detecting breathing disruptions during sleep includes a fastening arrangement for attaching a detecting arrangement to the human body, having at least one microphone for detecting breathing sounds and a logic arrangement configured to evaluate the breathing sounds.

### FIELD OF THE INVENTION

The present invention relates to a medical detection device for detecting breathing disruptions during sleep.

### BACKGROUND INFORMATION

Obstructive sleep apnea is the most common form of sleep-related breathing disruptions. According to estimates, more than 8% of the population of Germany suffers from this disease and thus also from its consequences. Of the 8% thus affected, only about 5% is diagnosed with the disease and treated.

In obstructive sleep apnea, a complete cessation of breathing occurs for a longer period of time due to a complete closure of the upper airways. By definition, apnea is a cessation of breathing lasting more than 10 seconds. Hypopnea is another form of breathing disruption. It involves a 50% loss of airflow. Prolonged cessations of breathing or hypopnea recur many times, so that the affected person's sleep becomes very fragmented. The consequences of obstructive sleep apnea are daytime drowsiness and diminished performance in the short term. In the long term, obstructive sleep apnea may result in high blood pressure, increased risk of a heart attack, impotence, etc.

In practice, the tentative diagnosis is usually made relatively late during a detailed history taken by the family doctor upon increased suffering by the patient. When nocturnal cessation of breathing is suspected, definitive and differential diagnoses are made by time-consuming and expensive polysomnographic monitoring in a sleep laboratory.

European patent document EP 1 797 846 A1 discusses a method and a device for treating sleep apnea. The device is configured in such a way that it exerts force on the neck of the user so that the neck is superextended in order to prevent closure of the airways.

### SUMMARY OF THE INVENTION

An object of the exemplary embodiments and/or exemplary methods of the present invention is to propose a cost-effective self-test for home use for preliminary assessment of breathing disruptions, in particular apnea and/or hypopnea.

This object may be achieved by using a medical detection device having the features described herein. Advantageous refinements of the exemplary embodiments and/or exemplary methods of the present invention are further described herein. All combinations of at least two features disclosed in the description, the figures, and further herein are within the scope of the exemplary embodiments and/or exemplary methods of the present invention.

The exemplary embodiments and/or exemplary methods of the present invention concerns a disposable medical detection device for the detection of sleep apnea having, on the one hand, a fastening arrangement using which the detection device may be attached to the human body, in particular to the neck or the thorax area and, on the other hand, having at least one microphone for detecting breathing sounds which may be evaluated via an integral logic arrangement of the detection device in regard to the occurrence of breathing disruptions, in particular sleep apnea and/or hypopnea. In other words, the logic arrangement of the medical detection device are configured in such a way that the signal analysis of the signals (breathing sounds) detected by the microphone is executable so that the medical detection device is usable at home as a self-test for preliminary assessment of breathing disruptions. The medical detection device may be attached to the body, in particular to the neck and/or thorax area with the help of the fastening arrangement before going to bed, whereupon the detection device performs automatic analysis or evaluation, if needed, upon manual activation. A specific embodiment is particularly advantageous, in which the at least one microphone is configured as a micro-electro-mechanical system (MEMS component) in order to minimize the weight of the detection device on the one hand, and to make wearing of the detection device as comfortable as possible for the user on the other hand. Alternatively configured miniature microphones may also be used in addition or as an alternative.

In a refinement of the exemplary embodiments and/or exemplary methods of the present invention it is, advantageously provided that the fastening arrangement includes an adhesive tape and/or are configured as adhesive tapes. In other words, a specific embodiment may be used in which the at least one microphone with a signal-conducting connection to the logic arrangement and the logic arrangement are integrated in an adhesive tape which may be easily affixed to a predefined body part of the user and then removed after evaluation.

One specific embodiment is particularly advantageous in which the logic arrangement are configured in such a way that the breathing sounds are analyzable by way of signal analysis in regard to breathing disruptions, in particular in regard to cessation of breathing lasting more than a minimum period of time, for example, for more than 10 seconds. Only apneas and/or hypopneas that extend beyond the predefined or predefinable minimum period may be counted and evaluated. The logic arrangement may include a breathing disruption counter which is incremented by one every time the logic arrangement detect a breathing disruption lasting longer than the minimum period of time.

One specific embodiment is particularly advantageous in which the cardiac activity, in particular the heart rate, of the affected person is taken into consideration by the logic arrangement for a plausibility check of potential breathing disruptions. This special logic arrangement configuration is based on the finding that when there is a longer-lasting breathing disruption, in particular cessations of breathing, an increase in the heart rate occurs due to hypoxia of the body. When breathing resumes, the heart rate becomes normal. The breathing disruption counter may be incremented by one only if the cardiac activity, in particular the heart rate, exceeds a certain value, for example a set value or a moving average during a potential breathing disruption, i.e., during the time period in which there is a noticeable breathing sound or lack thereof.

In an advantageous refinement of the exemplary embodiments and/or exemplary methods of the present invention it is provided that for monitoring or evaluating the cardiac activity, the logic arrangement evaluates heart sounds which are detected via the at least one microphone. In other words, in the course of the signal analysis process performed by the logic arrangement, the heart sounds, particularly in regard to the heart rate, may be evaluated in addition to breathing sounds for a plausibility check of potential breathing disruptions.

As mentioned above, a specific embodiment may be used in which the logic arrangement count the number of breathing disruptions lasting longer than a minimum period of time. The logic arrangement may be configured in such a way that they evaluate a breathing disruption as apnea and/or hypopnea only when the heart rate exceeds a certain value during a time period in which there is a noticeable breathing sound or lack thereof.

It is particularly advantageous if an output arrangement is associated with the logic arrangement for outputting an evaluation result. For example, it is possible to configure the output arrangement in such a way that they indicate if there is a minimum number of breathing disruptions, for example one breathing disruption or five breathing disruptions, during sleep. The output arrangement may also be configured in such a way, for example, that they provide information about the number of the detected, which may be validated, apneas and/or hypopneas. Thus, for example, different outputs of the logic arrangement may be activated depending on the final count of the breathing disruption counter resulting in different outputs.

In addition or alternatively, the output arrangement may include a wireless arrangement via which wireless retrieval of the evaluation results and/or the state of the breathing disruption counter is possible. In addition or alternatively to the wireless arrangement, it is conceivable to provide a connection for cable-supported readout of the evaluation results and/or the breathing disruption counter.

One specific embodiment of the medical detection device is advantageous in particular in which the output arrangement is configured to visually output an evaluation result based on an electrochemical reaction. The power required by the output arrangement may thus be minimized. For example, an electrochemical color reaction may be induced via the voltage applied to the output of the logic arrangement which may be a function of the number of the breathing disruptions detected and, in particular, verified by a plausibility check, which colors a certain area of the detection device, the adhesive tape in particular, which may be in an irreversible way. In a further variant, different outputs of the logic arrangement may be activated depending on the final count of the breathing disruption counter which then trigger color reactions in different areas of the detection device, in particular of the adhesive tape, thereby coloring the detection device in different areas and which may be in an irreversible way. For example, a marking made visible by an electrochemical reaction, in particular on the adhesive tape, may provide information about the number of breathing disruptions, in particular apneas and hypopneas verified by a plausibility check in particular.

One specific embodiment is particularly advantageous in which the detection device has an integral voltage source supplying the logic arrangement and/or the microphone and/or the output arrangement with the necessary electric power. It is particularly advantageous if the voltage source includes a capacitor or a capacitor system so that the detection device could be disposed of after use without risk to the environment.

In a refinement of the exemplary embodiments and/or exemplary methods of the present invention, it is advantageously provided that the microphone may be situated in the detection device, in particular in the adhesive tape, in such a way that in the case of a detection device attached to humans, i.e., their bodies, the microphone rests directly on the skin, which may be on the neck, particularly which may be close to the carotid artery and/or on the thorax in order to detect breathing and/or heart sounds. It is also conceivable to use separate microphones to detect breathing and heart sounds.

Further advantages, features and details of the present invention are derived from the following description of an exemplary embodiment and from the drawing.

### BRIEF DESCRIPTION OF THE DRAWING

FIG. 1 shows a schematic representation of a medical detection device.

### DETAILED DESCRIPTION

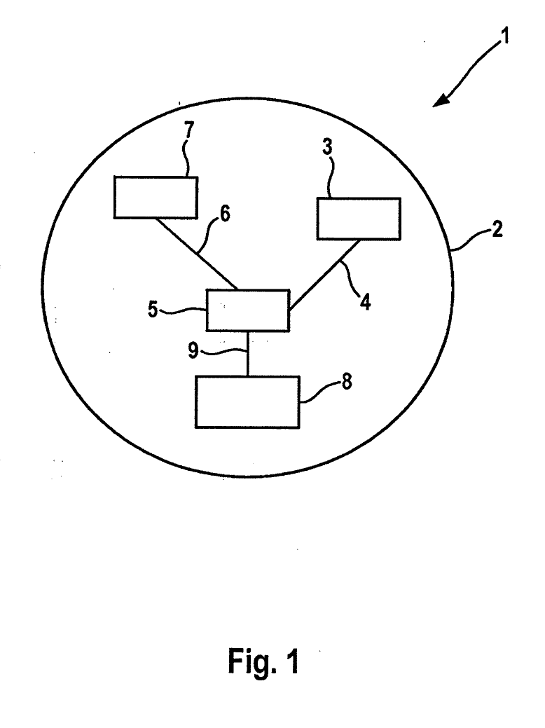

FIG. 1 shows a highly schematic illustration of a possible specific embodiment of a medical detection device 1 for detecting breathing disruptions during sleep. Detection device 1 includes a fastening arrangement 2 configured as adhesive tapes for detachably attaching detection device 1 on the skin of a human body. The adhesive tape has adhesive areas facing the body (not shown).

Detection device 1 includes a microphone 3 configured as a MEMS component for recording, i.e., detecting breathing and heart sounds. Microphone 3 is connected to a logic arrangement 5 including a microprocessor via signal line 4 for conducting signals. Logic arrangement 5 is supplied via a supply line 6 with electric power from an integral voltage source 7 of detection device 1, the electric power being transmitted by logic arrangement 5 to supply microphone 3 and by output arrangement 8 connected to logic arrangement 5 for conducting signals.

Logic arrangement 5 is configured to evaluate the breathing sounds ascertained by microphone 3 and to monitor and evaluate the detected heart sounds. If no breathing sound or a noticeable breathing sound is detected during a predefined period of time, ten seconds, for example, logic arrangement 5 recognizes a potential sleep disruption. If, at the same time, during the time breathing sounds are detected, the heart rate, derived by logic arrangement 5 from the heart sounds, increases above a predefined value, a set value or a moving average value, for example, sleep apnea is verified by a plausibility check and an integral breathing disruption counter of logic arrangement 5 is incremented by one. If necessary, the evaluation of the heart sounds and plausibility check of potential sleep apnea may be dispensed with. In this case, the breathing disruption counter may be incremented by one if an apnea and/or hypopnea lasts longer than a predefined period of time.

As mentioned above, logic arrangement 5 is connected to output arrangement 8 for conducting signals. A signal line 9 serves this purpose. In a very simple embodiment variant, a voltage is applied to signal line 9 of logic arrangement 5 as soon as the breathing disruption counter exceeds a certain value, such as 1, 5, or 10. This voltage activates output arrangement 8 in such a way that the output arrangement triggers an electrochemical color reaction which irreversibly colors a certain area of fastening arrangement 2 configured as adhesive tapes. In an alternative variant, different outputs of logic arrangement 5, the microprocessor in particular, may be activated as a function of the content of the breathing disruption counter, these outputs being connected to different color reaction areas so that depending on the activated output, different areas of the signal arrangement are colored. The number of detected breathing disruptions during sleep or a range thereof may be visualized for the user via color coding or a display.

1-10. (canceled)

11. A medical detection device for detecting breathing disruptions during sleep, comprising:
a detecting arrangement having at least one microphone for detecting breathing sounds and having a logic arrangement configured to evaluate the breathing sounds; and
a fastening arrangement for securing the detecting arrangement to the human body.

12. The detection device of claim 11, wherein the fastening arrangement includes an adhesive tape.

13. The detection device of claim 11, wherein the logic arrangement is configured to evaluate the breathing sounds in terms of occurrence of at least one of an apnea and a hypopnea lasting longer than a minimum period of time.

14. The detection device of claim 13, wherein the logic arrangement is configured to consider cardiac activity, in particular the heart rate, to perform a plausibility check of breathing disruption, in particular cessation of breathing.

15. The detection device of claim 13, wherein the logic arrangement is configured to monitor the cardiac activity on the basis of heart sounds detected via the microphone.

16. The detection device of claim 13, wherein the logic arrangement is configured to count the breathing disruptions verified by a plausibility check lasting a minimum period of time.

17. The detection device of claim 11, wherein an output arrangement is assigned to the logic arrangement for outputting an evaluation result.

18. The detection device of claim 17, wherein the output arrangement is configured to display an evaluation result based on an electrochemical reaction.

19. The detection device of claim 11, further comprising:
a voltage source, including at least one capacitor.

20. The detection device of claim 11, wherein the microphone is situated so that in the case of a detection arrangement attached to the body, the microphone rests directly on the skin.

21. The detection device of claim 11, wherein the microphone is situated so that in the case of a detection arrangement attached to the body, the microphone rests directly on the neck.

22. The detection device of claim 11, wherein the microphone is situated so that in the case of a detection arrangement attached to the body, the microphone rests directly on at least one of the neck close the carotid artery and the thorax.
